(19) United States
(12) Patent Application Publication
(10) Pub. No.: US 2020/0205766 A1
(43) Pub. Date: Jul. 2, 2020

(54) SYSTEMS AND METHODS FOR CONTROLLING MEDICAL RADIATION EXPOSURE TO PATIENTS

(71) Applicants: SHANGHAI UNITED IMAGING INTELLIGENCE CO., LTD., Shanghai (CN); SHANGHAI UNITED IMAGING HEALTHCARE CO., LTD., Shanghai (CN)

(72) Inventors: Dijia WU, Shanghai (CN); Yongqin XIAO, Shanghai (CN)

(73) Assignees: SHANGHAI UNITED IMAGING INTELLIGENCE CO., LTD., Shanghai (CN); SHANGHAI UNITED IMAGING HEALTHCARE CO., LTD., Shanghai (CN)

(21) Appl. No.: 16/729,401

(22) Filed: Dec. 29, 2019

(30) Foreign Application Priority Data
Dec. 29, 2018 (CN) 201811639074.4

(51) Int. Cl. A61B 6/00; G06K 9/62; G06K 9/20; G06K 9/00; A61B 6/03

(52) U.S. Cl. CPC A61B 6/541; G06K 9/6256; G06K 9/2018; G06K 9/00342; G06K 2209/055; A61B 6/5294; A61B 6/505; A61B 6/032; A61B 6/5247; A61B 6/542

(57) ABSTRACT

A method for exposure controlling in medical device may include obtaining one or more exposure parameters relating to an exposure process associated with an object performed by a radiation device. The method may also include obtaining object information relating to the object. The method may also include determining an exposure moment based on the object information. The method may also include causing the radiation device to perform the exposure process to the object based on the one or more exposure parameters and the exposure moment.

### CROSS-REFERENCE TO RELATED APPLICATIONS

This application claims priority to Chinese Patent Application No. 201811639074.4 filed on Dec. 29, 2018, the entire contents of which are incorporated herein by reference.

### TECHNICAL FIELD

The present disclosure generally relates to medical technology, and more particularly, systems and methods for exposure controlling in medical radiation.

### BACKGROUND

The medical radiation device usually performs an exposure process based on exposure timing information defined by various components inside the medical radiation device. The patient, which is the most important factor, however, is not considered during the entire exposure process. Even though a patient's real-time video is acquired through a visualization device, the exposure moment is determined based on the technician's observation of the patient's position, posture, and motion state shown in the video. As such, the errors and delays caused by the human in the determination of the exposure moment may lead to a low image quality, and even reimaging, resulting in a patient's exposure to more radiation rays. Therefore, it is desirable to provide systems and/or methods for automatically determining the exposure moment.

### SUMMARY

According to an aspect of the present disclosure, a system for exposure controlling in medical radiation may include one or more storage devices, and one or more processors configured to communicate with the one or more storage devices. The one or more storage devices may include a set of instructions. Optionally, the one or more storage devices may further include preset data for exposure controlling in medical radiation. When the one or more processors executing the set of instructions, the one or more processors may be directed to perform one or more of the following operations. The one or more processors may obtain one or more exposure parameters relating to an exposure process associated with an object performed by a radiation device. The one or more processors may obtain object information relating to the object. The one or more processors may determine an exposure moment based on the object information. The one or more processors may cause the radiation device to perform the exposure process to the object based on the one or more exposure parameters and the exposure moment.

In some embodiments, the object information may include at least one of a position of the object, a posture of the object, and a motion state of the object.

In some embodiments, to obtain the object information relating to the object, the one or more processors may obtain image information of the object. The one or more processors may obtain a first trained machine learning model. The one or more processors may obtain the object information by processing the image information using the first trained machine learning model.

In some embodiments, the image information may be provided by an image capture device.

In some embodiments, to obtain the object information related to the object, the one or more processors may determine skeleton information of the object based on the image information of the object. The one or more processors may determine the at least one of the position of the object, the posture of the object, and the motion state of the object based on the skeleton information.

In some embodiments, the skeleton information of the object may be determined using the first trained machine learning model based on the image information of the object.

In some embodiments, the first trained machine learning model is provided by: obtaining sample image information relating to a plurality of sample objects; obtaining mark points and body vectors of the plurality of sample objects in the sample image information, each body vector linking two of the mark points; and obtaining the first trained machine learning model by training a preliminary model using the mark points and the vectors of the sample object.

In some embodiments, the first trained machine learning model may be a neural network.

In some embodiments, to determine the exposure moment based on the object information, the one or more processors may determine whether the at least one of the position of the object, the posture of the object, and the motion state of the object satisfies a preset condition. In response to a determination that the at least one of the position of the object, the posture of the object, and the motion state of the object satisfies the preset condition, the one or more processors may determine the exposure moment.

In some embodiments, a determination result as to whether the at least one of the position of the object, the posture of the object, and the motion state of the object satisfies the preset condition may be obtained using a second trained machine learning model.

In some embodiments, the motion state of the object may include at least one of a motion state of the object's body and a respiration state of the object.

According to another aspect of the present disclosure, a method for exposure controlling in medical radiation may include one or more of the following operations. One or more processors may obtain one or more exposure parameters relating to an exposure process associated with an object performed by a radiation device. The one or more processors may obtain object information relating to the object. The one or more processors may determine an exposure moment based on the object information. The one or more processors may cause the radiation device to perform the exposure process to the object based on the one or more exposure parameters and the exposure moment.

According to yet another aspect of the present disclosure, a system for exposure controlling in medical radiation may include an obtaining module configured to obtain one or more exposure parameters relating to an exposure process associated with an object performed by a radiation device, and obtain object information relating to the object. The system may also include an exposure moment determination module configured to determine an exposure moment based on the object information. The system may also include an exposure module configured to cause the radiation device to perform the exposure process to the object based on the one or more exposure parameters and the exposure moment.

According to yet another aspect of the present disclosure, a non-transitory computer readable medium may include at least one set of instructions for exposure controlling in medical radiation. Optionally, the non-transitory computer readable medium may further include preset data for exposure controlling in medical radiation. The at least one set of instructions may be executed by one or more processors of a computer server. The one or more processors may obtain one or more exposure parameters relating to an exposure process associated with an object performed by a radiation device. The one or more processors may obtain object information relating to the object. The one or more processors may determine an exposure moment based on the object information. The one or more processors may cause the radiation device to perform the exposure process to the object based on the one or more exposure parameters and the exposure moment.

According to yet another aspect of the present disclosure, a system for exposure controlling in medical radiation may include one or more image capture devices configured to obtain image information of an object. The image information may be processed to determine object information of the object. The object information may be processed to determine an exposure moment at which a radiation device perform an exposure process to the object.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities and combinations set forth in the detailed examples discussed below.

### BRIEF DESCRIPTION OF THE DRAWINGS

The present disclosure is further described in terms of exemplary embodiments. These exemplary embodiments are described in detail with reference to the drawings. These embodiments are non-limiting exemplary embodiments, in which like reference numerals represent similar structures throughout the several views of the drawings, and wherein:

FIG. 1 is a schematic diagram illustrating an exemplary medical radiation system according to some embodiments of the present disclosure;

FIG. 2 is a schematic diagram illustrating hardware and/or software components of an exemplary computing device according to some embodiments of the present disclosure;

FIG. 3 is a schematic diagram illustrating hardware and/or software components of an exemplary mobile device according to some embodiments of the present disclosure;

FIG. 4 is a block diagram illustrating an exemplary processing device according to some embodiments of the present disclosure;

FIG. 5 is a block diagram illustrating an exemplary obtaining module according to some embodiments of the present disclosure;

FIG. 6 is a flowchart illustrating an exemplary process for exposure controlling in medical radiation according to some embodiments of the present disclosure;

FIG. 7 is a flowchart illustrating an exemplary process for determining skeleton information of an object according to some embodiments of the present disclosure;

FIG. 8 is a flowchart illustrating an exemplary process for determining an exposure moment according to some embodiments of the present disclosure;

FIG. 9 is a flowchart illustrating an exemplary process for generating a skeleton identification model according to some embodiments of the present disclosure;

FIG. 10 is a schematic diagram illustrating an exemplary network structure of a convolutional neural network VGG-16 model according to some embodiments of the present disclosure;

FIG. 11 is a schematic diagram illustrating an exemplary network structure of a convolutional neural network ResNet model according to some embodiments of the present disclosure;

FIG. 12 is a schematic diagram illustrating an exemplary iterative convolutional neural network according to some embodiments of the present disclosure;

FIG. 13 is a schematic diagram illustrating exemplary mark points of an object according to some embodiments of the present disclosure;

FIG. 14 is schematic diagrams illustrating an exemplary image including two persons according to some embodiments of the present disclosure;

FIG. 15 is schematic diagrams illustrating an exemplary body vector according to some embodiments of the present disclosure; and

FIGS. 16-19 are schematic diagrams illustrating exemplary skeleton maps indicating different postures according to some embodiments of the present disclosure.

### DETAILED DESCRIPTION

In the following detailed description, numerous specific details are set forth by way of examples in order to provide a thorough understanding of the relevant disclosure. However, it should be apparent to those skilled in the art that the present disclosure may be practiced without such details. In other instances, well-known methods, procedures, systems, components, and/or circuitry have been described at a relatively high-level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but to be accorded the widest scope consistent with the claims.

The terminology used herein is for the purpose of describing particular example embodiments only and is not intended to be limiting. As used herein, the singular forms “a,” “an,” and “the” may be intended to include the plural forms as well, unless the context clearly indicates otherwise. It will be further understood that the terms “comprise,” “comprises,” and/or “comprising,” “include,” “includes,” and/or “including,” when used in this specification, specify the presence of stated features, integers, steps, operations, elements, and/or components, but do not preclude the presence or addition of one or more other features, integers, steps, operations, elements, components, and/or groups thereof.

It will be understood that the term “system,” “engine,” “unit,” “module,” and/or “block” used herein are one method to distinguish different components, elements, parts, section or assembly of different level in ascending order. However, the terms may be displaced by other expression if they achieve the same purpose.

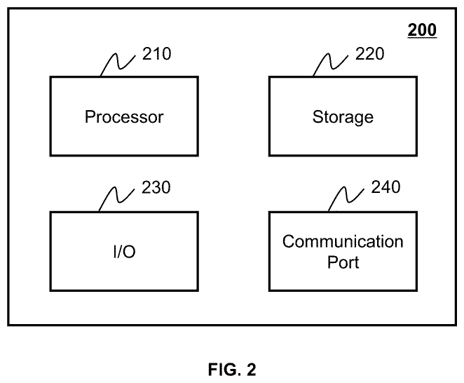

Generally, the word “module,” “unit,” or “block,” as used herein, refers to logic embodied in hardware or firmware, or to a collection of software instructions. A module, a unit, or a block described herein may be implemented as software and/or hardware and may be stored in any type of non-transitory computer-readable medium or other storage device. In some embodiments, a software module/unit/block may be compiled and linked into an executable program. It will be appreciated that software modules can be callable from other modules/units/blocks or from themselves, and/or may be invoked in response to detected events or interrupts. Software modules/units/blocks configured for execution on computing devices (e.g., processor 210 as illustrated in FIG. 2) may be provided on a computer-readable medium, such as a compact disc, a digital video disc, a flash drive, a magnetic disc, or any other tangible medium, or as a digital download (and can be originally stored in a compressed or installable format that needs installation, decompression, or decryption prior to execution). Such software code may be stored, partially or fully, on a storage device of the executing computing device, for execution by the computing device. Software instructions may be embedded in a firmware, such as an EPROM. It will be further appreciated that hardware modules/units/blocks may be included in connected logic components, such as gates and flip-flops, and/or can be included of programmable units, such as programmable gate arrays or processors. The modules/units/blocks or computing device functionality described herein may be implemented as software modules/units/blocks, but may be represented in hardware or firmware. In general, the modules/units/blocks described herein refer to logical modules/units/blocks that may be combined with other modules/units/blocks or divided into sub-modules/sub-units/sub-blocks despite their physical organization or storage. The description may be applicable to a system, an engine, or a portion thereof.

It will be understood that when a unit, engine, module or block is referred to as being “on,” “connected to,” or “coupled to,” another unit, engine, module, or block, it may be directly on, connected or coupled to, or communicate with the other unit, engine, module, or block, or an intervening unit, engine, module, or block may be present, unless the context clearly indicates otherwise. As used herein, the term “and/or” includes any and all combinations of one or more of the associated listed items.

These and other features, and characteristics of the present disclosure, as well as the methods of operation and functions of the related elements of structure and the combination of parts and economies of manufacture, may become more apparent upon consideration of the following description with reference to the accompanying drawings, all of which form a part of this disclosure. It is to be expressly understood, however, that the drawings are for the purpose of illustration and description only and are not intended to limit the scope of the present disclosure. It is understood that the drawings are not to scale.

For illustration purposes, the following description is provided to help better understanding a process for exposure controlling. It is understood that this is not intended to limit the scope of the present disclosure. For persons having ordinary skills in the art, a certain amount of variations, changes and/or modifications may be deducted under the guidance of the present disclosure. Those variations, changes and/or modifications do not depart from the scope of the present disclosure.

The present disclosure provides systems and/or methods for controlling medical radiation exposure to objects. The systems and/or methods may obtain object information indicating at least a current state of an object. The object information may include at least one of location information of the object, a posture of the object, and a motion state of the object (e.g., a motion state of the object's body, and/or a respiration state of the object). The systems and/or methods may obtain the object information based on one or more sensors. Alternatively or additionally, the systems and/or methods may obtain the object information based on image information of the object acquired by one or more image information acquisition devices. For example, the systems and/or methods may identify, using a trained skeleton identification model (e.g., a convolutional neural network), skeleton information of the object based on the image information. The systems and/or methods may determine the object information based on the skeleton information. The systems and/or methods may automatically determine an exposure moment based on the object information. The systems and/or methods may cause a radiation device (e.g., a medical imaging device) to perform an exposure process (e.g., a medical imaging process) at the exposure moment. The exposure moment may be automatically determined using an artificial intelligence operation, which may reduce the workload of a technician, reduce errors and delays caused by human in the determination of the exposure moment, improving the image quality, reducing the probability of reimaging, and protecting the patient from unnecessary radiation exposure. The skeleton information may be determined based on a convolutional neural network, which may improve the efficiency and the accuracy of determining the exposure moment.

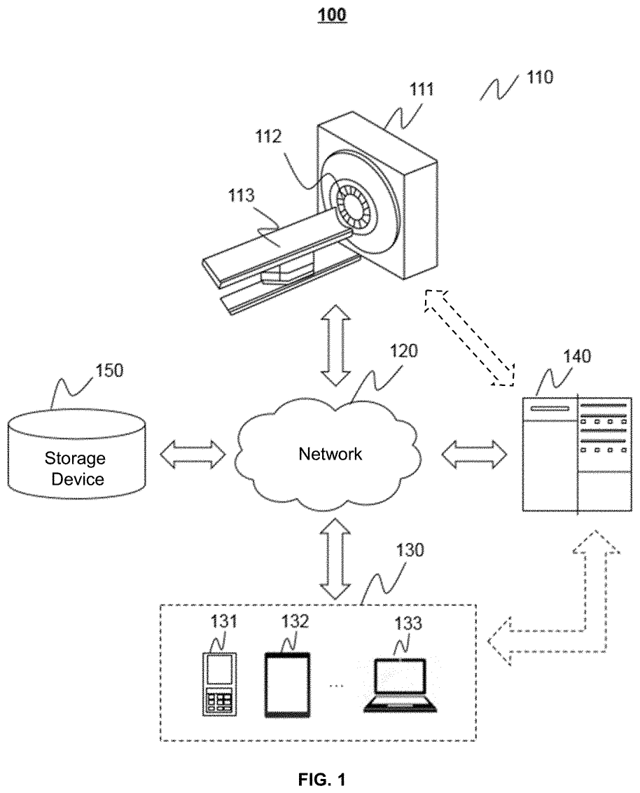

FIG. 1 is a schematic diagram illustrating an exemplary medical radiation system according to some embodiments of the present disclosure. In some embodiments, the medical radiation system 100 may be applied to any application scenario in which radiation rays are used for medical diagnosis, generating images, and/or providing a treatment, such as a computed tomography (CT) system, a digital radiography (DR) system, a C-arm X-ray system, a computed tomography-positron emission tomography (CT-PET) system, a nuclear magnetic resonance-computed tomography (NMR-CT), a radiotherapy system, or the like, or a combination thereof.

As illustrated in FIG. 1, the medical radiation system 100 may include a radiation device 110, a network 120, one or more terminals 130, a processing device 140, and a storage device 150. The components in the medical radiation system 100 may be connected in one or more of various ways. Merely by way of example, the radiation device 110 may be connected to the processing device 140 through the network 120. As another example, the radiation device 110 may be connected to the processing device 140 directly as indicated by the bi-directional arrow in dotted lines linking the radiation device 110 and the processing device 140. As a further example, the storage device 150 may be connected to the processing device 140 directly or through the network 120. As still a further example, the terminal 130 may be connected to the processing device 140 directly (as indicated by the bi-directional arrow in dotted lines linking the terminal 130 and the processing device 140) or through the network 120.

In some embodiments, the radiation device 110 may include an imaging device, a treatment device, or the like, or any combination thereof. The imaging device may include a computed tomography (CT) scanner, a digital radiography (DR) scanner, a C-arm X-ray scanner, a digital substraction angiography (DSA) scanner, a dynamic spatial reconstructor (DSR) scanner, an X-ray microscopy scanner, a multi-modality scanner, or the like, or a combination thereof. Exemplary multi-modality scanners may include a computed tomography-positron emission tomography (CT-PET) scanner, a computed tomography-magnetic resonance imaging (CT-MRI) scanner, etc. The treatment device may include a linear accelerator, a cyclotron, a synchrotron, etc., configured to perform a radio therapy on an object.

In some embodiments, the radiation device 110 may include a gantry 111, a radiation source 112, and a scanning table 113. The radiation source 112 may emit radiation rays to the object that is placed on the scanning table 113. The radiation rays may include X-rays, y-rays, a-rays, ultraviolet, laser, neutron, proton, or the like, or a combination thereof.

In some embodiments, if the radiation device 110 includes an imaging device, the radiation device 110 may further include a detector (not shown in FIG. 1). The detector and the radiation source 112 may be oppositely mounted on the gantry 111. An object may be placed on the scanning table 113 and moved into a detection tunnel (e.g., a space between the detector and the radiation source 112) of the radiation device 110. The object may be biological or non-biological. Merely by way of example, the object may include a patient, a man-made object, etc. As another example, the object may include a specific portion, organ, and/or tissue of the patient. For example, the object may include head, brain, neck, body, shoulder, arm, thorax, cardiac, stomach, blood vessel, soft tissue, knee, feet, or the like, or any combination thereof. In the present disclosure, “subject” and “object” are used interchangeably.

The detector may receive the radiation rays passed through the object. In some embodiments, the detector may include a plurality of detector units, which may be arranged in a channel direction and a row direction. The detector may include a scintillation detector (e.g., a cesium iodide detector) or a gas detector.

In some embodiments, the medical radiation system 100 may further include one or more object information acquisition devices configured to acquire object information related to the object. The object information may indicate at least a current state of the object.

In some embodiments, the object information acquisition device may include one or more image information acquisition devices configured to acquire image information of the object. The image information acquisition device may include a visible light camera, an infrared camera, or the like. In some embodiments, the image information acquisition device may be integrated in the radiation device 110 (e.g., the gantry 111). In some embodiments, the image information of the object may include one or more infrared images, one or more visible light images, or the like, or any combination thereof.

In some embodiments, the object information acquisition device may include one or more location acquisition devices configured to acquire location information of the object. The location acquisition device may include one or more sensors (e.g., a laser-ranging sensor, an infrared sensor, a pressure sensor, etc.) with a positioning function. In some embodiments, the image information acquisition device may be integrated in the radiation device 110 (e.g., the gantry 111 and/or the scanning table 113).

In some embodiments, the image acquisition device and/or the location acquisition device may be placed outside the medical radiation system 100, e.g., any locations outside the medical radiation system 100. In some embodiments, the image acquisition device and/or the location acquisition device may be placed outside the medical radiation system 100 and communicate with the medical radiation system 100.

In some embodiments, the object information acquisition device may include one or more posture acquisition devices configured to acquire a posture of the object. The posture acquisition device may include one or more sensors (e.g., a posture sensor, a pyroelectric infrared sensor, a pressure sensor, etc.) that may be placed in one or more locations (e.g., head, chest, abdomen, arms, legs, etc.) on the object to mark and identify the posture of the object.

In some embodiments, the object information acquisition device may include one or more respiration sensors (e.g., an air flow sensor, a thorax sensor, and/or an air pressure sensor) configured to detect a respiration state of the object. The respiration sensor may acquire one or more parameters related to the respiration of the object, such as the respiration flow capacity, the respiration flow direction, air pressure, the variation of object's thorax, or the like. In some embodiments, the respiration sensor may be placed on the object (e.g., on the abdomen, around nostrils, and/or around the mouth). For example, the air flow sensor and/or the air pressure sensor may be placed on a breathing mask worn by the object. As another example, the thorax sensor may be a slice placed on the chest of the object or a belt around the chest of the object.

The network 120 may facilitate exchange of information and/or data. In some embodiments, one or more components of the medical radiation system 100 (e.g., the radiation device 110, the terminal 130, the processing device 140, the storage device 150, or the object information acquisition device) may send information and/or data to another component(s) in the medical radiation system 100 via the network 120. For example, the processing device 140 may obtain, via the network 120, image information of the object from the object information acquisition device. As another example, the processing device 140 may obtain a user instruction from the terminal 130 via the network 120. As still another example, the processing device 140 may obtain scan data from the radiation device 110 via the network 120. In some embodiments, the network 120 may be any type of wired or wireless network, or combination thereof. The network 120 may be and/or include a public network (e.g., the Internet), a private network (e.g., a local area network (LAN), a wide area network (WAN)), etc.), a wired network (e.g., an Ethernet network), a wireless network (e.g., an 802.11 network, a Wi-Fi network), a cellular network (e.g., a Long Term Evolution (LTE) network), a frame relay network, a virtual private network (“VPN”), a satellite network, a telephone network, routers, hubs, switches, server computers, and/or any combination thereof. Merely by way of example, the network 120 may include a cable network, a wireline network, an optical fiber network, a telecommunications network, an intranet, an Internet, a local area network (LAN), a wide area network (WAN), a wireless local area network (WLAN), a metropolitan area network (MAN), a wide area network (WAN), a public telephone switched network (PSTN), a Bluetooth™ network, a ZigBee™ network, a near field communication (NFC) network, or the like, or any combination thereof. In some embodiments, the network 120 may include one or more network access points. For example, the network 120 may include wired or wireless network access points such as base stations and/or internet exchange points through which one or more components of the medical radiation system 100 may be connected to the network 120 to exchange data and/or information.

The terminal 130 include a mobile device 130-1, a tablet computer 130-2, a laptop computer 130-3, or the like, or any combination thereof. In some embodiments, the mobile device 130-1 may include a smart home device, a wearable device, a smart mobile device, a virtual reality device, an augmented reality device, or the like, or any combination thereof. In some embodiments, the smart home device may include a smart lighting device, a control device of an intelligent electrical apparatus, a smart monitoring device, a smart television, a smart video camera, an interphone, or the like, or any combination thereof. In some embodiments, the wearable device may include a bracelet, footgear, eyeglasses, a helmet, a watch, clothing, a backpack, an accessory, or the like, or any combination thereof. In some embodiments, the smart mobile device may include a smartphone, a personal digital assistant (PDA), a gaming device, a navigation device, a point of sale (POS) device, or the like, or any combination thereof. In some embodiments, the virtual reality device and/or the augmented reality device may include a virtual reality helmet, a virtual reality glass, a virtual reality patch, an augmented reality helmet, an augmented reality glass, an augmented reality patch, or the like, or any combination thereof. For example, the virtual reality device and/or the augmented reality device may include a Google Glass, an Oculus Rift, a HoloLens, a Gear VR, etc. In some embodiments, the terminal 130 may remotely operate the radiation device 110. In some embodiments, the terminal 130 may operate the radiation device 110 via a wireless connection. In some embodiments, the terminal 130 may receive information and/or instructions inputted by a user, and send the received information and/or instructions to the radiation device 110 or to the processing device 140 via the network 120. In some embodiments, the terminal 130 may receive data and/or information from the processing device 140. In some embodiments, the terminal 130 may be part of the processing device 140. In some embodiments, the terminal 130 may be omitted.

In some embodiments, the processing device 140 may process data obtained from the radiation device 110, the terminal 130, the storage device 150, or the object information acquisition device. In some embodiments, the processing device 140 may obtain image information to train a preset prediction model. For example, the processing device 140 may obtain a skeleton identification model that can identify human skeleton information by training a preliminary machine model based on the obtained image information of a patient. In some embodiments, the processing device 140 may obtain object information from the object information acquisition device and determine an exposure moment based on the object information. The processing device 140 may be a central processing unit (CPU), a digital signal processor (DSP), a system on a chip (SoC), a microcontroller unit (MCU), or the like, or any combination thereof.

In some embodiments, the processing device 140 may be a single server or a server group. The server group may be centralized or distributed. In some embodiments, the processing device 140 may be local or remote. For example, the processing device 140 may access information and/or data stored in the radiation device 110, the terminal 130, the object information acquisition device, and/or the storage device 150 via the network 120. As another example, the processing device 140 may be directly connected to the radiation device 110, the terminal 130, the object information acquisition device, and/or the storage device 150, to access stored information and/or data. In some embodiments, the processing device 140 may be implemented on a cloud platform. Merely by way of example, the cloud platform may include a private cloud, a public cloud, a hybrid cloud, a community cloud, a distributed cloud, an inter-cloud, a multi-cloud, or the like, or any combination thereof. In some embodiments, the processing device 140 may be implemented on a computing device 200 having one or more components illustrated in FIG. 2 in the present disclosure.

The storage device 150 may store data and/or instructions. In some embodiments, the storage device 150 may store data obtained from the terminal 130 and/or the processing device 140. For example, the storage device 150 may store one or more images obtained from the object information acquisition device. In some embodiments, the storage device 150 may store data and/or instructions that the processing device 140 may execute or use to perform exemplary methods described in the present disclosure. For example, the storage device 150 may store preset data (e.g., including one or more preset images, one or more preset exposure parameters used by the radiation device to perform an exposure process to an object, one or more preset conditions used to determine an exposure moment, etc.) and/or instructions that the processing device 140 may execute or use to automatically determine an exposure moment and/or cause the radiation device to perform an exposure process to an object. In some embodiments, the storage device 150 may include a mass storage, removable storage, a volatile read-and-write memory, a read-only memory (ROM), or the like, or any combination thereof. Exemplary mass storage may include a magnetic disk, an optical disk, a solid-state drive, etc. Exemplary removable storage may include a flash drive, a floppy disk, an optical disk, a memory card, a zip disk, a magnetic tape, etc. Exemplary volatile read-and-write memory may include a random-access memory (RAM). Exemplary RAM may include a dynamic RAM (DRAM), a double date rate synchronous dynamic RAM (DDR SDRAM), a static RAM (SRAM), a thyristor RAM (T-RAM), and a zero-capacitor RAM (Z-RAM), etc. Exemplary ROM may include a mask ROM (MROM), a programmable ROM (PROM), an erasable programmable ROM (PEROM), an electrically erasable programmable ROM (EEPROM), a compact disk ROM (CD-ROM), and a digital versatile disk ROM, etc. In some embodiments, the storage device 150 may be implemented on a cloud platform. Merely by way of example, the cloud platform may include a private cloud, a public cloud, a hybrid cloud, a community cloud, a distributed cloud, an inter-cloud, a multi-cloud, or the like, or any combination thereof.

In some embodiments, the storage device 150 may be connected to the network 120 to communicate with one or more components of the medical radiation system 100 (e.g., the radiation device 110, the object information acquisition device, the terminal 130, the processing device 140). One or more components of the medical radiation system 100 may access the data or instructions stored in the storage device 150 via the network 120. In some embodiments, the storage device 150 may be directly connected to or communicate with one or more components of the medical radiation system 100 (e.g., the terminal 130, the processing device 140). In some embodiments, the storage device 150 may be part of the processing device 140.

FIG. 2 is a schematic diagram illustrating hardware and/or software components of an exemplary computing device according to some embodiments of the present disclosure. As illustrated in FIG. 2, the computing device 200 may include a processor 210, a storage 220, an input/output (I/O) 230, and a communication port 240. In some embodiments, the processing device 140 and/or the terminal 130 may be implemented on the computing device 200.

The processor 210 may execute computer instructions (program code) and, when executing the instructions, cause the processing device 140 to perform functions of the processing device 140 in accordance with techniques described herein. The computer instructions may include, for example, routines, programs, objects, components, signals, data structures, procedures, modules, and functions, which perform particular functions described herein. In some embodiments, the processor 210 may process data and/or images obtained from the radiation device 110, the terminal 130, the storage device 150, and/or any other component of the medical radiation system 100. For example, the processor 210 may obtain object information and determine an exposure moment based on the object information. In some embodiments, the processor 210 may include one or more hardware processors, such as a microcontroller, a microprocessor, a reduced instruction set computer (RISC), an application specific integrated circuits (ASICs), an application-specific instruction-set processor (ASIP), a central processing unit (CPU), a graphics processing unit (GPU), a physics processing unit (PPU), a microcontroller unit, a digital signal processor (DSP), a field programmable gate array (FPGA), an advanced RISC machine (ARM), a programmable logic device (PLD), any circuit or processor capable of executing one or more functions, or the like, or any combinations thereof.

Merely for illustration, only one processor is described in the computing device 200. However, it should be noted that the computing device 200 in the present disclosure may also include multiple processors. Thus operations and/or method steps that are performed by one processor as described in the present disclosure may also be jointly or separately performed by the multiple processors. For example, if in the present disclosure the processor of the computing device 200 executes both process A and process B, it should be understood that process A and process B may also be performed by two or more different processors jointly or separately in the computing device 200 (e.g., a first processor executes process A and a second processor executes process B, or the first and second processors jointly execute processes A and B).

The storage 220 may store data/information obtained from the radiation device 110, the terminal 130, the storage device 150, or any other component of the medical radiation system 100. In some embodiments, the storage 220 may include a mass storage device, removable storage device, a volatile read-and-write memory, a read-only memory (ROM), or the like, or any combination thereof. For example, the mass storage may include a magnetic disk, an optical disk, a solid-state drive, etc. The removable storage may include a flash drive, a floppy disk, an optical disk, a memory card, a zip disk, a magnetic tape, etc. The volatile read-and-write memory may include a random access memory (RAM). The RAM may include a dynamic RAM (DRAM), a double date rate synchronous dynamic RAM (DDR SDRAM), a static RAM (SRAM), a thyristor RAM (T-RAM), and a zero-capacitor RAM (Z-RAM), etc. The ROM may include a mask ROM (MROM), a programmable ROM (PROM), an erasable programmable ROM (PEROM), an electrically erasable programmable ROM (EEPROM), a compact disk ROM (CD-ROM), and a digital versatile disk ROM, etc. In some embodiments, the storage 220 may store one or more programs and/or instructions to perform exemplary methods described in the present disclosure. For example, the storage 220 may store a program (e.g., in the form of computer-executable instructions) for the processing device 140 for automatically determining an exposure moment.

The I/O 230 may input or output signals, data, and/or information. In some embodiments, the I/O 230 may enable user interaction with the processing device 140. In some embodiments, the I/O 230 may include an input device and an output device. Exemplary input devices may include a keyboard, a mouse, a touch screen, a microphone, or the like, or a combination thereof. Exemplary output devices may include a display device, a loudspeaker, a printer, a projector, or the like, or a combination thereof. Exemplary display devices may include a liquid crystal display (LCD), a light-emitting diode (LED)-based display, a flat panel display, a curved screen, a television device, a cathode ray tube (CRT), or the like, or a combination thereof.

The communication port 240 may be connected to a network (e.g., the network 120) to facilitate data communications. The communication port 240 may establish connections between the processing device 140 and the radiation device 110, the terminal 130, or the storage device 150. The connection may be a wired connection, a wireless connection, or combination of both that enables data transmission and reception. The wired connection may include an electrical cable, an optical cable, a telephone wire, or the like, or any combination thereof. The wireless connection may include Bluetooth, Wi-Fi, WiMAX, WLAN, ZigBee, mobile network (e.g., 3G, 4G, 5G, etc.), or the like, or a combination thereof. In some embodiments, the communication port 240 may be a standardized communication port, such as RS232, RS485, etc. In some embodiments, the communication port 240 may be a specially designed communication port. For example, the communication port 240 may be designed in accordance with the digital imaging and communications in medicine (DICOM) protocol.

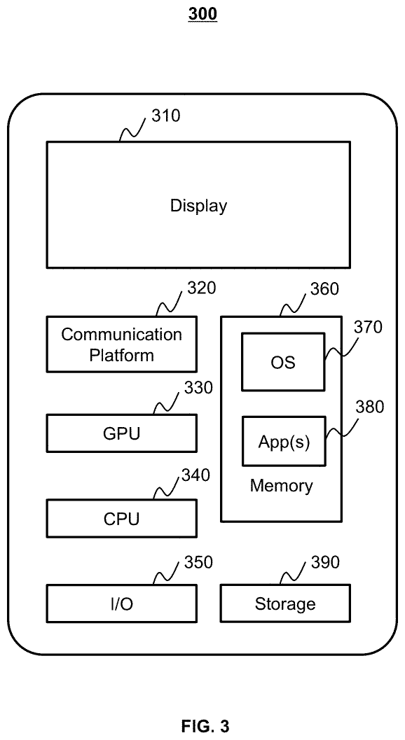

FIG. 3 is a schematic diagram illustrating hardware and/or software components of a mobile device according to some embodiments of the present disclosure. In some embodiments, the processing device 140 and/or the terminal 130 may be implemented on the computing device 200. As illustrated in FIG. 3, the mobile device 300 may include a display 310, a communication platform 320, a graphic processing unit (GPU) 330, a central processing unit (CPU) 340, an I/O 350, a memory 360, and a storage 390. In some embodiments, any other suitable component, including but not limited to a system bus or a controller (not shown), may also be included in the mobile device 300. In some embodiments, a mobile operating system 370 (e.g., iOS, Android, Windows Phone, etc.) and one or more applications 380 may be loaded into the memory 360 from the storage 390 in order to be executed by the CPU 340. The applications 380 may include a browser or any other suitable mobile apps for receiving and rendering information relating to image processing or other information from the processing device 140. User interactions with the information stream may be achieved via the I/O 350 and provided to the processing device 140 and/or other components of the medical radiation system 100 via the network 120.

To implement various modules, units, and their functionalities described in the present disclosure, computer hardware platforms may be used as the hardware platform(s) for one or more of the elements described herein, The hardware elements, operating systems and programming languages of such computers are conventional in nature, and it is presumed that those skilled in the art are adequately familiar therewith to adapt those technologies to control exposure in medical radiation as described herein. A computer with user interface elements may be used to implement a personal computer (PC) or another type of work station or terminal device, although a computer may also act as a server if appropriately programmed. It is believed that those skilled in the art are familiar with the structure, programming and general operation of such computer equipment and as a result, the drawings should be self-explanatory.

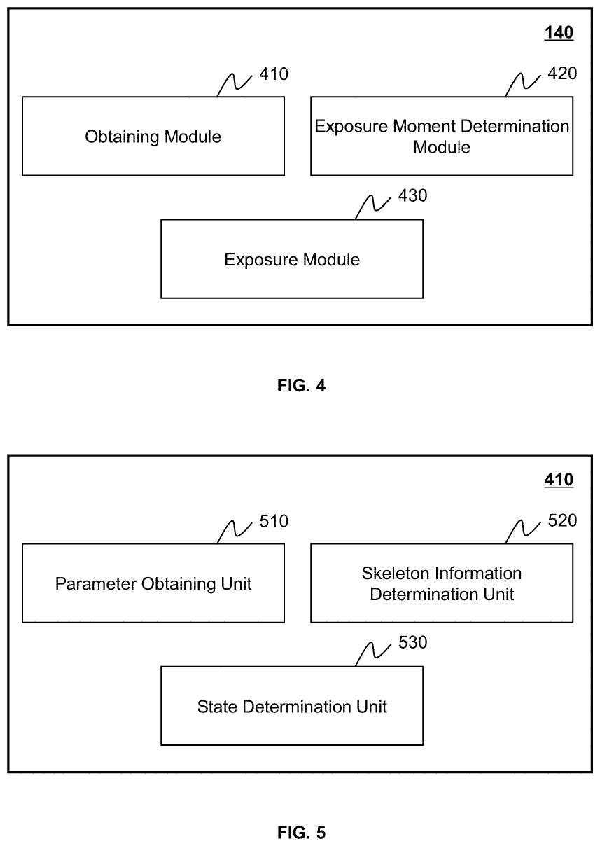

FIG. 4 is a schematic diagram illustrating an exemplary processing device 140 according to some embodiments of the present disclosure. The processing device 140 may include an obtaining module 410, an exposure moment determination module 420, and an exposure module 430. At least a portion of the processing device 140 may be implemented on the computing device 200 as illustrated in FIG. 2 or the mobile device 300 as illustrated in FIG. 3.

The obtaining module 410 may obtain one or more exposure parameters relating to an exposure process associated with an object performed by a radiation device (e.g., the radiation device 110).

The obtaining module 410 may further obtain object information relating to the object. The object information may indicate at least a current state of the object. The object information may include at least one of location information of the object, a posture of the object, and a motion state of the object.

The exposure moment determination module 420 may determine an exposure moment based on the object information, In some embodiments, the exposure moment may refers to a time when the radiation source 112 of the radiation device 110 emits radiation rays to the object during a treatment process or a time when the radiation source 112 emits radiation rays to the object and the detector of the radiation device 110 is charged during an imaging process.

The exposure module 430 may cause the radiation device 110 to perform the exposure process to the object based on the one or more exposure parameters and the determined exposure moment. For example, the exposure module 430 may cause the radiation device 110 to emit radiation rays to the object according to the one or more exposure parameters at the exposure moment to perform a treatment process. As another example, the exposure module 430 may cause the radiation device 110 to emit radiation rays to the object and cause the detector to be charged according to the one or more exposure parameters at the exposure moment to perform an imaging process. Further, at the exposure moment, according to the one or more exposure parameters, the medical radiation system 100 may generate high-voltage signals through a high-voltage generator to initiate the radiation source 112 (e.g., a bulb tube) to emit radiation rays. At the same time, the medical radiation system 100 may initiate an ionization chamber and the detector of the radiation device 110 to receive the radiation rays that go through the object, thereby obtaining one or more medical images (e.g., a CT image, a DR image, or the like, or any combination thereof) of the object.

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. For example, the processing device 140 may further include a storage module (not shown in FIG. 4). The storage module may be configured to store data generated during any process performed by any component of in the processing device 140. As another example, each of components of the processing device 140 may include a storage device. Additionally or alternatively, the components of the processing device 140 may share a common storage device.

FIG. 5 is a block diagram illustrating an exemplary obtaining module according to some embodiments of the present disclosure. In some embodiments, the obtaining module 410 may include a parameter obtaining unit 510, a skeleton information determination unit 520, and a state determination unit 530.

The parameter obtaining unit 510 may obtain one or more exposure parameters relating to an exposure process associated with an object performed by a radiation device (e.g., the radiation device 110).

The skeleton information determination unit 520 may determine skeleton information of the object based on the image information of the object.

In some embodiments, the skeleton information may include a plurality of mark points related to the object, a plurality of body vectors related to the object, a skeleton map, a surface model of the object, or the like, or any combination thereof. In some embodiments, the mark point may represent a component of the object.

The skeleton information determination unit 520 may determine body vectors based on the plurality of mark points. In some embodiments, the skeleton information determination unit 520 may determine the skeleton map by connecting the mark points based on the body vectors.

The state determination unit 530 may determine at least one of the location information of the object, the posture of the object, and the motion state of the object based on the skeleton information.

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure.

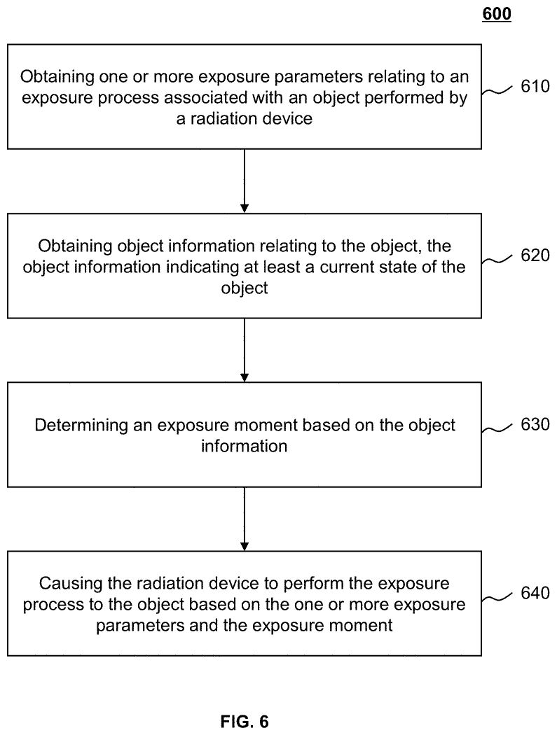

FIG. 6 is a flowchart illustrating an exemplary process for exposure controlling in medical radiation according to some embodiments of the present disclosure. In some embodiments, one or more operations of the process 500 illustrated in FIG. 6 may be implemented in the medical radiation system 100 illustrated in FIG. 1. For example, the process 500 illustrated in FIG. 6 may be stored in a storage medium (e.g., the storage device 150, and/or the storage 220) of the medical radiation system 100 in the form of instructions, and invoked and/or executed by the processing device 140 (e.g., the processor 210 of the computing device 200 as illustrated in FIG. 2, the CPU 340 of the mobile device 300 as illustrated in FIG. 3, or one or more modules/units of the processing device 140 illustrated in FIGS. 4-5). The operations of the illustrated process presented below are intended to be illustrative. In some embodiments, the process 500 may be accomplished with one or more additional operations not described, and/or without one or more of the operations discussed. Additionally, the order in which the operations of the process 500 as illustrated in FIG. 6 and described below is not intended to be limiting.

In some embodiments, the process 600 may include: obtaining one or more exposure parameters relating to an exposure process associated with an object performed by a radiation device (operation 610); obtaining object information relating to the object, the object information indicating at least a current state of the object (operation 620); determining an exposure moment based on the object information (operation 630); and causing the radiation device to perform the exposure process to the object based on the one or more exposure parameters and the exposure moment (operation 640).

In 610, the processing device 140 (e.g., the obtaining module 410 and/or the parameter obtaining unit 510) may obtain one or more exposure parameters relating to an exposure process associated with an object performed by a radiation device (e.g., the radiation device 110).

In some embodiments, the exposure process may refer to a treatment process using radiation rays and/or an imaging process for taking one or more medical images (e.g., X-ray images, CT images, etc.) using radiation rays.

In some embodiments, the exposure parameter may include an exposure intensity, an exposure duration, an exposure position, or the like, or any combination thereof. The exposure intensity may refer to the intensity of radiation rays emitted to the object. The exposure duration may refer to a duration for emitting radiation rays to the object. The exposure position may refer to a region that radiation rays emitted from the radiation source 112 cover.

In some embodiments, the exposure parameter may be determined based on a radiation plan of the object (e.g., a patient) before the exposure process are performed. The radiation plan of the object may include, for example, the gender, the age, the symptom type, the disease type, the historical medical record of the object, a treatment plan, an imaging protocol, or the like, or any combination thereof. For example, patient A is 40 years old and the exposure intensity may be set as a first value. Patient B is 70 years old and the exposure intensity may be set as a second value different from (e.g., less than) the first value. As another example, patient C's symptom type is a brain tumor, and the exposure duration may be set as a third value. Patient D's symptom type is a lung tumor, and the exposure duration may be set as a fourth value different from (e.g., greater than) the third value. In some embodiments, the processing device 140 may automatically determine the one or more exposure parameters. In some embodiments, a user of the medical radiation system 100 (e.g., a doctor or a technician) may set the one or more exposure parameters.

In some embodiments, the exposure parameter may be adjusted according to the object's condition in real time. For example, after a stage of a treatment performed to a diseased target (e.g., a tumor) in the object by the radiation device 110, the size of the diseased target may be reduced. At the subsequent stage of the treatment performed to the diseased target, a collimator of the radiation device 110 may be adjusted to adjust the exposure position to protect normal issue around the diseased target from being damaged by radiation rays.

In 620, the processing device 140 (e.g., the obtaining module 410 and/or the parameter obtaining unit 510) may obtain object information relating to the object. The object information may indicate at least a current state of the object. The object information may include at least one of location information of the object, a posture of the object, and a motion state of the object.

In some embodiments, the location information of the object may include an actual space location of the object and/or a location of the object on the scanning table 113. In some embodiments, the processing device 140 may obtain the location information of the object based on the location acquisition device and/or images of the object captured by the image information acquisition device.

In some embodiments, the processing device 140 may obtain image information of the object captured by the object information acquisition device, for example, a camera, and determine the object's real-time location information using a machine learning algorithm. In some embodiments, the processing device 140 may obtain a plurality of images captured from different angles of the object. Each of the plurality of images may be captured by a camera. Further, for each of the plurality of images, the processing device 140 may identify a position of the object in the image using a machine learning algorithm, such as a trained neural network model. For example, the position of the object in the image may indicate that the object is located in a rectangular region with dimensions of 40 mm by 20 mm in the image. The upper border of the rectangular region is 10 mm from the upper border of the image, and the right border of the rectangular region is 10 mm from the right border of the image. The processing device 140 may determine, based on the location of the object in the image, a space location of the object including an angle relative to the camera capturing the image. For example, the processing device 140 may determine a direction that the object locates at relative to the camera (e.g., the object is locates in the north of the camera) based on the location of the rectangle area in the image. The different images of an object at a space location may be captured by the cameras set at different angles. The processing device 140 may determine straight lines based on an actual location and angle of the different cameras and determine the space location including angles that is the actual location of the object. That is, the intersection point of the straight lines corresponding to the different camera may be designated as the actual space location of the object.

In some embodiments, the processing device 140 may determine the actual location of the object by reconstructing a three-dimensional (3D) space model based on the plurality of images taken from different angles of the object. For example, the processing device 140 may perform 3D reconstruction from stereo images based on camera calibration, e.g., two or more cameras that are calibrated by one or more parameters (e.g., the focal length, the optical center, or the distortion factor).

In some embodiments, the plurality of image taken from different angles of the object may be taken at the same time (e.g., the current time).

In some embodiments, the processing device 140 may obtain one or more images captured by the image information acquisition device. The processing device 140 may determine a position of the object on the scanning table 113 by processing the one or more images taken by cameras set at different angles using an image processing algorithm. In some embodiments, the image processing algorithm may include binarization, histogram processing, brightness mapping, an addition operation, a flip operation, a ruler degree operation, a logarithm operation, an exponential operation, a template convolution operation, an filter operation (e.g., mean filter, maximum filter, minimum filter), anisotropic diffusion, morphological operations (e.g., expansion and corrosion), force field transformation, or the like, or any combination thereof. That is, the processing device 140 may determine the actual space locations of skeleton joints of a patient based on the images taken by the cameras set at different angles. Accordingly, it may be determined whether a target treatment object is located in a suitable exposure area. In the meantime, the posture of the patient or the target treatment object may be determined. For example, it may be determined whether the left arm of the patient is placed flat on the table.

In some embodiments, a single image information acquisition device may be placed on the gantry 111, and a reference object may be set in the view of the single image information acquisition device (e.g., one or more marks may be set on the scanning table 113). The processing device 140 may determine the location of the object on the scanning table 113 by comparing a location of the object in an image captured by the single image information acquisition device to a location of the reference object in the image.

In some embodiments, the processing device 140 may obtain the posture of the object based on the posture acquisition device and/or images of the object captured by the image information acquisition device. For example, the processing device 140 may obtain the posture by identifying the object in one or more images taken by the image information acquisition device using the image processing algorithm. In some embodiments, different patients may correspond to different postures.

In some embodiments, the processing device 140 may obtain the motion state of the object based on at least one of the location information, the posture of the object, the respiration sensor, and images of the object captured by the image information acquisition device.

In some embodiments, the processing device 140 may obtain a plurality of successive frames taken by the image information acquisition device (e.g., a camera) during a period of time (e.g., 0.5 s, 1 s, 2 s, 5 s, 10 s, etc.). The processing device 140 may determine the location information of the object corresponding to the plurality of successive frames. The processing device 140 may determine the variation of the location information, e.g., the motion distance, of the object in the period of time based on the location information of the object corresponding to the plurality of successive frames.

In some embodiments, the processing device 140 may obtain the variation of the pressure distribution in the scanning table 113 from the pressure sensor positioned at the scanning table 113 in a period of time. The processing device 140 may determine the motion state of the object based on the variation of the pressure distribution in a period of time.

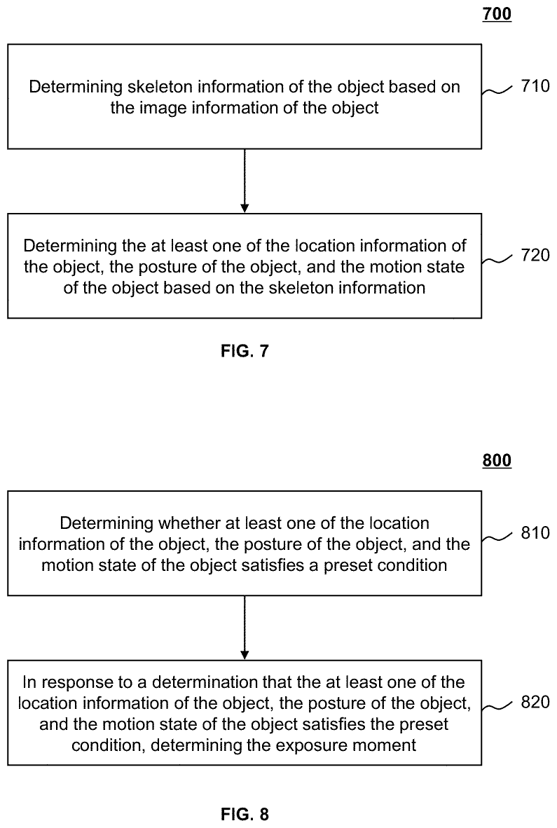

In some embodiments, the processing device 140 may determine skeleton information of the object by processing one or more images taken by the image information acquisition device. The processing device 140 may determine the location information, the posture, or the motion state of the object based on the skeleton information. More details related to the determination of the skeleton information may be found elsewhere in the present disclosure (e.g., description in connection with FIG. 7).

In some embodiments, the processing device 140 may determine the motion state of the object by determining a respiration state of the object based on the respiratory sensor. For example, when the processing device 140 receives, from the air flow sensor, a positive value of air flow of which the absolute value is larger than a value threshold (e.g., 10% of the maximum exhalation and/or inspiration of the object), the processing device 140 may determine that the object is in an exhalation state. When the processing device 140 receives, from the air flow sensor, a negative value of air flow of which the absolute value is larger than the value threshold, the processing device 140 may determine that the object is in an inspiratory state. When the processing device 140 receives, from the air flow sensor, a value of air flow of which the absolute value is less than or equal to the value threshold, the processing device 140 may determine that the object is in a state of holding breath (e.g., a state between exhalation and inspiratory).

As another example, when the processing device 140 receives, from the air pressure sensor, a value above the normal atmospheric pressure, the processing device 140 may determine that the object is in an exhalation state. When the processing device 140 receives, from the air pressure sensor, a value below the normal atmospheric pressure, the processing device 140 may determine that the object is in an inspiratory state. When the processing device 140 receives, from the air pressure sensor, a value equal to the normal atmospheric pressure, the processing device 140 may determine that the object is in a state of holding breath (e.g., a state between exhalation and inspiratory).

As still another example, when the processing device 140 receives, from the thorax sensor, a value of the thorax size that is between a maximum thorax size and a minimum thorax size of the object, the processing device 140 may determine that the object is in the exhalation state or the inspiratory state. When the processing device 140 receives, from the thorax sensor, a value of the thorax size that is equal to the maximum thorax size or the minimum thorax size of the object, the processing device 140 may determine that the object is in a state of holding breath (e.g., a state between exhalation and inspiratory).

In some embodiments, the processing device 140 may determine the respiration state based on the image information acquired by the image information acquisition device. In some embodiments, the processing device 140 may identify the variation (e.g., ups and downs) of the thorax based on real-time images captured by the image information acquisition device and determine the respiration state based on the variation of the thorax. In some embodiments, the processing device 140 may identify the outline of the object or a component (e.g., thorax) of the object in a plurality of successive frames, and determine the respiration state by tracking location of the outline in plurality of successive frames. In some embodiments, the processing device 140 may identify the variation (e.g., ups and downs) of the thorax based on the image information. For example, the processing device 140 may determine one or more mark points corresponding to a component (e.g., thorax) of the object. The processing device 140 may determine the respiration state by tracking the location of the one or more mark points in a plurality of successive frames.

In some embodiments, the object information of the object may relate to a component of the object based on the radiation plan. For example, if the radiation plan indicates an imaging process performed to the head of the object (e.g., a patient), the processing device 140 may obtain the object information related to the head of the object, such as, the location information of the head of the object, the posture of the head of the object, and the motion state of the head of the object. The location information, the posture, and the motion state of other components, such as the legs, the hands of the object may not be considered. As another example, if the radiation plan indicates an imaging process performed to the abdomen of the object, the processing device 140 may obtain the object information related to the abdomen of the object, such as, the location information of the abdomen of the object, the posture of the abdomen of the object, and the motion state of the abdomen of the object. In some embodiments, for different objects (e.g., different patients), the location information, the posture, and the motion state that is needed to determine may be different.

In some embodiment, the location information of the object and the posture of the object may correspond to a time point (e.g., the current time), The motion state of the object may indicate whether the object or at least a component of the object is moving during a period of time from a past time point (e.g., prior to the current time) to the current time.

In 630, the processing device 140 (e.g., the exposure moment determination module 420) may determine an exposure moment based on the object information. In some embodiments, the exposure moment may refers to a time when the radiation source 112 of the radiation device 110 emits radiation rays to the object during a treatment process or a time when the radiation source 112 emits radiation rays to the object and the detector of the radiation device 110 is charged during an imaging process.

In some embodiments, the processing device 140 may determine whether the at least one of the location information of the object, the posture of the object, and the motion state of the object satisfies a preset condition. In response to a determination that the at least one of the location information of the object, the posture of the object, and the motion state of the object satisfies the preset condition, the processing device 140 may determine the exposure moment. For example, the processing device 140 may determine the current time as the exposure moment.

For example, when a patient's leg is injured and a CT scan is needed to perform to the patient's injured leg, the processing device 140 may cause the radiation device 110 to perform the exposure process (e.g., the CT scan) when the processing device 140 determines that the patient is at a first posture (e.g., a supine posture). When a patient's back is injured and a CT scan is needed to perform to the patient's injured back, the processing device 140 may cause the radiation device 110 to perform the exposure process (e.g., the CT scan) when the processing device 140 determines that the patient is at a second posture (e.g., a prostrate posture) different from the first posture.

After determining a patient's location, body posture and motion state, the processing device 140 may determine whether the location is within a preset range, whether the patient's posture is the supine posture, whether the patient is in a static state, or the like, or any combination thereof.

More details related to the determination of the exposure moment may be found elsewhere in the present disclosure (e.g., the description in connection with FIG. 8).

In 640, the processing device 140 (e.g., the exposure module 430) may cause the radiation device 110 to perform the exposure process to the object based on the one or more exposure parameters and the determined exposure moment. For example, the processing device 140 may cause the radiation device 110 to emit radiation rays to the object according to the one or more exposure parameters at the exposure moment to perform a treatment process. As another example, the processing device 140 may cause the radiation device 110 to emit radiation rays to the object and cause the detector to be charged according to the one or more exposure parameters at the exposure moment to perform an imaging process. Further, at the exposure moment, according to the one or more exposure parameters, the medical radiation system 100 may generate high-voltage signals through a high-voltage generator to initiate the radiation source 112 (e.g., a bulb tube) to emit radiation rays. At the same time, the medical radiation system 100 may initiate an ionization chamber and the detector of the radiation device 110 to receive the radiation rays that go through the object, thereby obtaining one or more medical images (e.g., a CT image, a DR image, or the like, or any combination thereof) of the object.

It should be noted that the above description is merely provided for the purposes of illustration, and not intended to limit the scope of the present disclosure, For persons having ordinary skills in the art, multiple variations and modifications may be made under the teachings of the present disclosure, However, those variations and modifications do not depart from the scope of the present disclosure. For example, under the premise that operation 610 is performed before operation 640, the processing device 140 may perform operation 610 before, after, or simultaneously with operation 620 and/or operation 630. As another example, the process 600 may include an operation before operation 630, in which the object information may be further processed to make the processing device 140 able to identify the condition of the object (e.g., the patient).

FIG. 7 is a flowchart illustrating an exemplary process for determining skeleton information of an object according to some embodiments of the present disclosure. In some embodiments, one or more operations of the process 700 illustrated in FIG. 7 may be implemented in the medical radiation system 100 illustrated in FIG. 1. For example, the process 700 illustrated in FIG. 7 may be stored in a storage medium (e.g., the storage device 150, and/or the storage 220) of the medical radiation system 100 in the form of instructions, and invoked and/or executed by the processing device 140 (e.g., the processor 210 of the computing device 200 as illustrated in FIG. 2, the CPU 340 of the mobile device 300 as illustrated in FIG. 3, or one or more modules/units of the processing device 140 illustrated in FIGS. 4-5), The operations of the illustrated process presented below are intended to be illustrative. In some embodiments, the process 700 may be accomplished with one or more additional operations not described, and/or without one or more of the operations discussed. Additionally, the order in which the operations of the process 700 as illustrated in FIG. 7 and described below is not intended to be limiting. In some embodiments, the processing device 140 may perform at least a part of operation 620 based on the process 700.

In 710, the processing device 140 (e.g., the obtaining module 410 and/or the skeleton information determination unit 520) may determine skeleton information of the object based on the image information of the object.

In some embodiments, the skeleton information may include a plurality of mark points related to the object, a plurality of body vectors related to the object, a skeleton map, a surface model of the object, or the like, or any combination thereof.

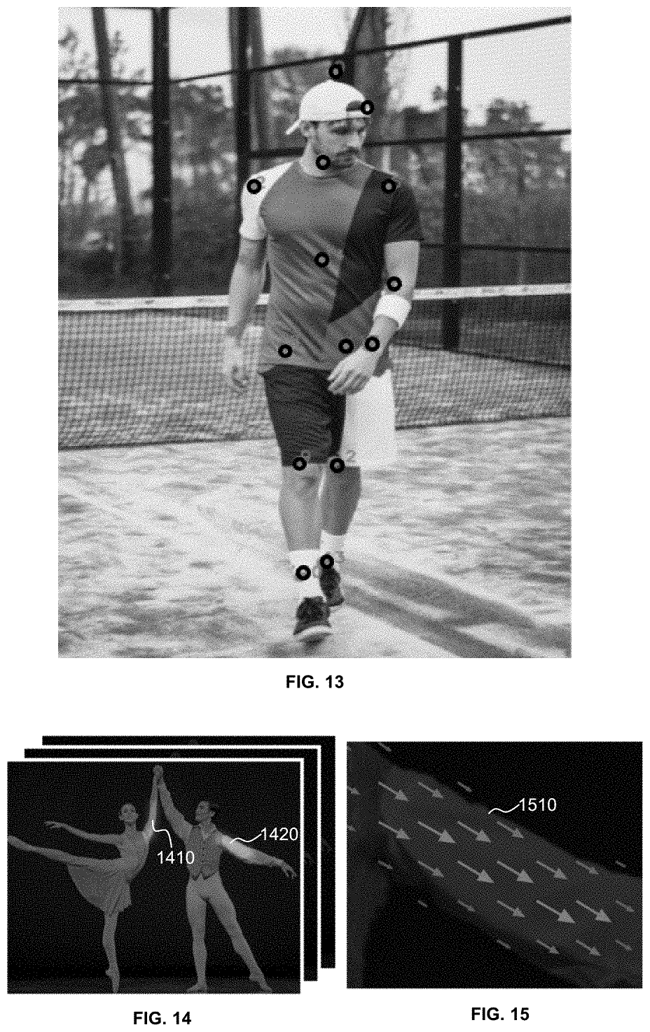

In some embodiments, the mark point may represent a component of the object. For example, the mark point may represent a joint of the object (e.g., as shown in FIG. 13), such as, the top of a head, a neck, a shoulder, an elbow, a wrist, a chest, a waist, a knee, an ankle, or the like. The body vector may be a part of a body vector field of the object's body. The body vector may indicate a direction and a distance between two of the plurality of mark points.

In some embodiments, the number (or count) and the distribution of the mark points may be determined based on the radiation plan. For example, the radiation plan may indicate a CT imaging process performed to the chest of the object. The number of the mark points representing the chest may be more than the number of the mark points representing other components of the object, e.g., the knees, the elbows, etc. Merely by way of example, the processing device 140 may determine three mark points to represent the chest of the object and determine one mark point located at the left knee to represent the left leg. As another example, the radiation plan may indicate a CT imaging process performed to the left leg of the object. The number of the mark points representing the left leg may be more than the number of the mark points representing other components of the object, e.g., the elbows, the right leg, the chest, etc. Merely by way of example, the processing device 140 may determine one mark point to represent the chest of the object and determine three mark points located at the top of the left leg, the left knee, and the left ankle, respectively, to represent the left leg.

FIG. 14 is schematic diagrams illustrating an exemplary image including two persons according to some embodiments of the present disclosure. As shown in FIG. 14, the woman in FIG. 14 includes a left upper arm 1410 and the man in FIG. 14 includes a left upper arm 1420. FIG. 15 is schematic diagrams illustrating an exemplary body vector according to some embodiments of the present disclosure. As shown in FIG. 15, the body vector 1510 in FIG. 15 represents the left upper arm 1420 in FIG. 14. The body vector 1510 indicates a direction from the left shoulder to the left elbow of the man. The body vector 1510 may include a plurality of sub-vectors, e.g., represented by the arrows in FIG. 15. In some embodiments, the magnitude of each sub-vector (e.g., represented by the lengths of the arrows in FIG. 15) may reflect the distance of pixels related to the sub-vector away from the line connecting the left shoulder and the left elbow of the man. The farther the distance is, the smaller the magnitude of the sub-vector may be. When the distance is greater than a distance threshold, the magnitude of the sub-vector may be 0. The determination of the body vector may improve the robustness of the determination of the skeleton information, especially improve the accuracy of connecting adjacent mark points that belong to the same object in the situation that there is more than one object in an image.

In some embodiments, the processing device 140 may determine the skeleton map by connecting the mark points based on the body vectors. In some embodiments, the processing device 140 may examine two of the plurality of mark points between which the distance is less than a distance threshold to determine whether the two mark points are matched points. The matched points may refer to two mark points that can be connected together to generate the skeleton map. The matched points may be considered as belonging to a same object. The processing device 140 may determine an average integral value along a line connecting the two mark points in the body vector field. The average integral value of the matched points may be relatively high. Therefore, the processing device 140 may divide the plurality of mark points into a plurality of pairs of matched points using a matching algorithm. A sum of the average integral values of the plurality of pairs of matched points may be maximum. The processing device 140 may determine the skeleton map by connecting the plurality of pairs of matched points. Exemplary matching algorithm may include a Hungarian algorithm, a maximum matching algorithm, a perfect matching algorithm, or the like.

Alternatively, the processing device 140 may connect two of the plurality of mark points between which the distance is less than the distance threshold and determine whether the connection is similar to one of the plurality of body vectors. In response to a determination that the connection is similar to one of the plurality of body vectors, the processing device 140 may determine the two mark points as the matched points.

Merely by way of example, the processing device 140 may identify three points 1-3 (e.g., representing the head, the neck, and the right shoulder, respectively) and two body vectors A (e.g., from the head to the neck) and B (e.g., from the neck to the right shoulder) in an image of the object. The processing device 140 may connect points 1 and 2, points 1 and 3, and points 2 and 3, respectively, and compare the lengths and the directions of the connections thereof to the lengths and the directions of the vectors A and B, respectively. The processing device 140 may determine that the connection between points 1 and 2 is similar to the vector A and the connection between points 2 and 3 is similar to the vector B. The processing device 140 may determine points 1 and 2 and points 2 and 3 as two pairs of matched points. The processing device 140 may determine the skeleton map of the object by connecting the points 1 and 2 and connecting the points 2 and 3. The determination that the connection between two mark points is similar to a vector may indicate that a difference between the length of the connection and that of the vector is less than a first threshold and an angle between the direction of the connection and that of the vector is less than a second threshold.

In some embodiments, the processing device 140 may determine the skeleton map using a trained machine learning model based on the mark points and the body vectors, For example, the mark points and the body vectors may be input into the trained machine learning model. The trained machine learning model may output the skeleton map.

In some embodiments, the processing device 140 may identify the mark points and the body vectors in an image of the object using a trained machine learning model (also referred to as a skeleton identification model). For example, the processing device 140 may determine features of the image of the object based on a preliminary feature extraction model. The processing device 140 may determine the mark points and the body vectors of the object based on the extracted features using the skeleton identification model (e.g., an iterative convolutional neural network (CNN), a recurrent neural network (RNN), a deep neural network (DNN), or the like, or any combination thereof),

In some embodiments, the surface model may be a 3D model. In some embodiments, the processing device 140 may determine the surface model of the object in an image based on dense pose. The pixels corresponding to the object in an image may be mapped to surface points using a trained machine learning model (e.g., a convolutional network) to determine the surface model of the object.

In some embodiments, the image information may be represented by an eigenvalue, such as the grayscale value, the color value, the texture eigenvalue, the edge feature, and the matrix, histogram, principal component obtained by transforming and/or processing the image information. In some embodiments, a feature map of the image information including image feature information may be obtained by extracting image features from the image information, for example, using a trained deep neural network with a body feature recognition capability and/or a feature extraction algorithm. FIG, 10 shows a network structure of a convolutional neural network VGG-16 model. FIG. 11 shows a network structure of a convolutional neural network ResNet model. In some embodiments, the feature information of the image information may be extracted using the trained VGG-16 convolution Neural Network model (e.g., see, K. Simonyan., et al. In ICLR, 2015) and/or the ResNet convolution Neural Network model (e.g., see, He K, Zhang X, Ren S, et al. Deep Residual Learning for Image Recognition [J]. Computer vision and pattern recognition, 2016: 770-778).

The feature map of the image information may be input into the skeleton identification model to identify the mark points and the body vectors. For example, the skeleton identification model may be an iterative convolutional neural network. The input to the iterative convolutional neural network may be the feature map F. F may be obtained by extracting image features from the image information. Mark points and body vectors may be obtained based on the trained skeleton identification model. For example, the trained skeleton identification model may output a probability map S including a plurality of mark points and a probability of each mark point for representing a component of the object. The trained skeleton identification model may also output a body vector field L of the object.

Merely by way of example, the output of the trained skeleton identification model may be represented by Equations (1) and (2) below:

S^{t+1}=ρ^{t}(F,S^{t}L^{t}) (1),

L^{t+1}=∅^{t}(F,S^{t},L^{t}) (2),

wherein, ρ^{t} refers to an inference function of an upper branch mark point identification network of the t^{th} CNN network of the iterative network architecture in FIG. 12; ∅^{t} refers to an inference function of a lower branch mark point identification network of the t^{th} CNN network of the iterative network architecture in FIG. 12; S^{t} refers to the probability map output by the previous network; L^{t} refers to the body vector field output by the previous network; S^{t+1} refers to the probability map output by the current network; L^{t+1}refers to the body vector field output by the current network. With the increase of the number of the iterative networks, the obtained probability map S and the body vector field L may be more accurate.

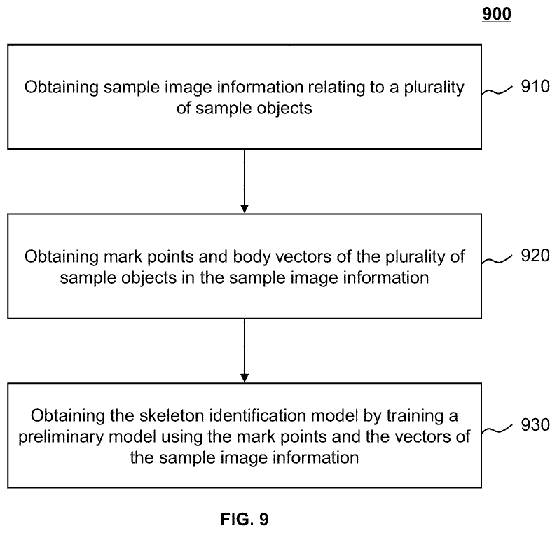

Details related to the generation of the trained machine learning model may be found elsewhere in the present disclosure (e.g., description in connection with FIG. 9).

In 720, the processing device 140 (e.g., the obtaining module 410 and/or the state determination unit 530) may determine at least one of the location information of the object, the posture of the object, and the motion state of the object based on the skeleton information.

In some embodiments, the processing device 140 may determine the location information based on the mark points. For example, the processing device 140 may determine a location of a mark point representing the head in an image. The processing device 140 may determine an actual space location of the head (e.g., the mark point) and/or a location of the head (e.g., the mark point) on the scanning table 113 based on the location of the mark point representing the head in the image. As another example, the actual location of the image information acquisition device (e.g., a camera) that acquires an image may be fixed. The processing device 140 may determine the location of the object in the image and determine the actual location of the object relative to the camera based on the location of the object in the image. More details related to the determination of the location information may be found elsewhere in the present disclosure (e.g., description in connection with operation 620 of the process 600 in FIG. 6).

In some embodiments, the processing device 140 may determine the posture of the object based on the skeleton map.

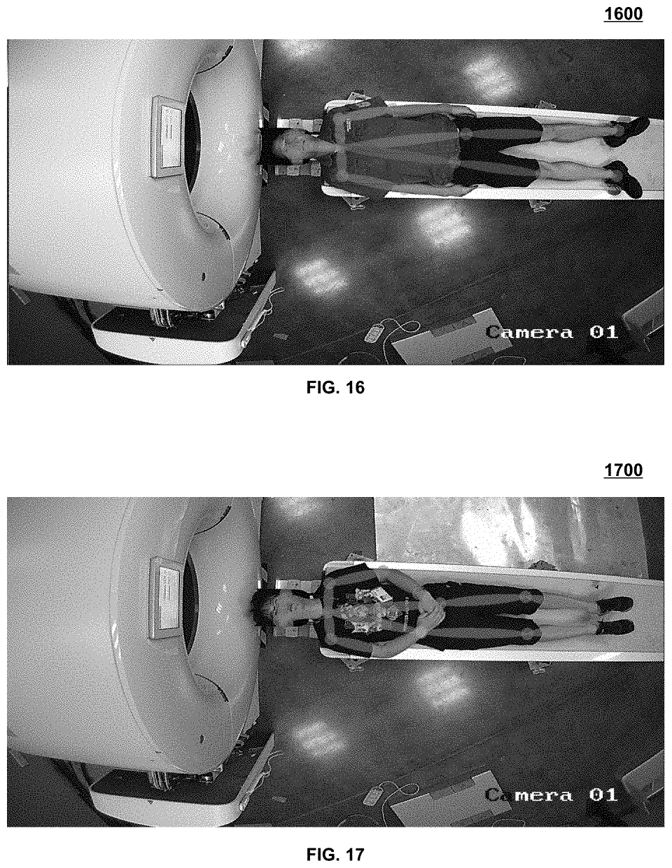

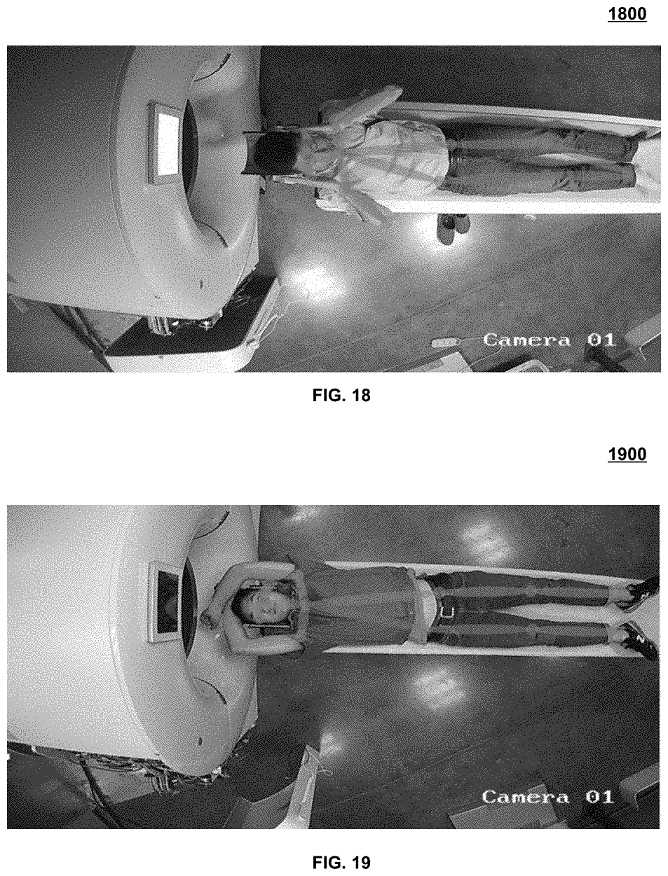

FIGS. 16-19 are schematic diagrams illustrating exemplary skeleton maps indicating different postures according to some embodiments of the present disclosure. As shown in FIGS. 16-19, different postures of the object may include different skeleton information (e.g., the locations of the mark points, the direction of the line connecting two mark points, etc.). As shown in FIG. 16, the skeleton map 1600 may indicate a posture that the object lies flat on the scanning table 113 and the object's hands are placed on both sides of the body. As shown in FIG. 17, the skeleton map 1700 may indicate a posture that the object lies flat on the scanning table 113 and the object's hands are stacked on the abdomen of the object. As shown in FIG. 18, the skeleton map 1800 may indicate a posture that the object lies flat on the scanning table 113 with hands on both sides of the head. As shown in FIG. 19, the skeleton map 1900 may indicate a posture that the object lies flat on the scanning table 113 with hands crossed over the top of the head. As shown in FIGS. 16-19, different postures may correspond to different skeleton maps.

In some embodiments, the processing device 140 may determine the posture of the object by comparing the skeleton map to a mask of a specific posture. For example, the processing device 140 may put the mask on the skeleton map and determine the area of overlapping parts between the mask and the skeleton map. In response to a determination that the area of overlapping parts is larger than or equal to an area threshold, the processing device 140 may determine that the skeleton map indicates a posture corresponding to the mask. In response to a determination that the area of overlapping parts is less than the area threshold, the processing device 140 may compare the skeleton map to other masks.

In some embodiments, the processing device 140 may determine the posture of the object based on the lines connecting the mark points in the skeleton map. For example, in the skeleton map corresponding to a supine posture, there may be a gap between the line representing the arm (e.g., the left arm and the right arm) and the line representing the trunk. As another example, in the skeleton map corresponding to a posture of lying on the side, at least a part of the line representing the arm (e.g., the left arm and the right arm) may overlap at least a part of the line representing the trunk. As still another example, the processing device 140 may determine the direction that the object faces at in a posture of lying on the side based on the bending direction of the line representing the leg (e.g., the left leg and/or the right leg) in the skeleton map.

In some embodiments, the posture of the object may be determined using a trained machine learning model based on the skeleton map. For example, the trained machine learning model may output one or more probabilities each of which corresponds to a posture. As another example, a relation between a display color of the skeleton map and the posture of the object may be determined in advance (e.g., a red color may correspond to the supine posture, a blue color may correspond to the posture of lying on the side, and a green color may correspond to the prostrate posture). The trained machine learning model may output a possible posture and the processing device 140 may display the skeleton map in a color corresponding to the posture.

In some embodiments, the processing device 140 may determine the motion state of the object based on the skeleton information. In some embodiments, the processing device 140 may obtain a plurality of successive frames taken by the image information acquisition device (e.g., a camera) during a period of time (e.g., 0.5 s, 1 s, 2 s, 5 s, 10 s, etc.). The processing device 140 may obtain the mark points in the plurality of successive frames. For each of the plurality of successive frames, the processing device 140 may determine the locations of the mark points in the frame. The processing device 140 may determine the variation of the locations of the mark points, e.g., the motion distance of each mark point, in the period of time based on the locations of the mark point in the plurality of successive frames.

In some embodiments, according to the radiation plan, the processing device may determine the motion state of a component of the object based on one or more marked points representing the component. For example, the radiation plan may indicate a CT imaging process performed to the right hand of the object. The processing device 140 may determine the motion distance of a mark point representing the right hand in the period of time based on the locations of the mark point in the plurality of successive frames. As another example, the radiation plan may indicate a CT imaging process performed to the right arm of the object. The processing device 140 may determine the motion distances of three mark points representing the right shoulder, the right elbow, and the right hand, respectively, in the period of time based on the locations of the mark points in the plurality of successive frames. If the processing device 140 determines that the right arm of the object is static, but the other components of the object are in a moving state (e.g., the patient is speaking or is shaking his/her head), the processing device 140 may still determine that the object or the right arm of the object is static.

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure.

FIG. 8 is a flowchart illustrating an exemplary process for determining an exposure moment according to some embodiments of the present disclosure. In some embodiments, one or more operations of the process 800 illustrated in FIG. 8 may be implemented in the medical radiation system 100 illustrated in FIG. 1. For example, the process 800 illustrated in FIG. 8 may be stored in a storage medium (e.g., the storage device 150, and/or the storage 220) of the medical radiation system 100 in the form of instructions, and invoked and/or executed by the processing device 140 (e.g., the processor 210 of the computing device 200 as illustrated in FIG. 2, the CPU 340 of the mobile device 300 as illustrated in FIG. 3, or one or more modules/units of the processing device 140 illustrated in FIGS. 4-5). The operations of the illustrated process presented below are intended to be illustrative. In some embodiments, the process 800 may be accomplished with one or more additional operations not described, and/or without one or more of the operations discussed. Additionally, the order in which the operations of the process 800 as illustrated in FIG. 8 and described below is not intended to be limiting. In some embodiments, the processing device 140 may perform operation 630 based on the process 800.

In 810, the processing device 140 (e.g., the exposure moment determination module 430) may determine whether at least one of the location information of the object, the posture of the object, and the motion state of the object satisfies a preset condition.

In some embodiments, the present condition may include a condition that the location information of the object is located at the exposure position, a condition that posture of the object is suitable for the radiation plan, a condition that the motion state of the object is static, a condition that the object is able to keep static for a certain period of time (e.g., 1 min) at the posture of the object, or the like, or any combination thereof.

For example, a CT scan is needed to perform to the head of a patient, the processing device 140 may determine the skeleton information of the patient through a skeleton identification model. The skeleton information may include the skeleton information of the head of the patient. Further, the processing device 140 may determine the location of the head of the patient based on the the skeleton information of the head of the patient. For example, the processing device 140 may determine the contour of the patient's head based on the skeleton information of the head of the patient. The processing device 140 may determine the location of the center point of the head based on the contour of the patient's head, and determine the location of the head based on the location of the center point of the head. Alternatively, the processing device 140 may determine the location of the top of the head based on the contour of the patient's head, and determine the location of the head based on the location of the top of the head.

After determining the location of the patient's head, the processing device 140 may whether the location of the patient's head is located at a preset range (e.g., the preset range may correspond to a preset location of the scanning table 113). In response to a determination that the location of the patient's head is located at the preset range, the processing device 140 may determine that the location of the patient's head satisfies the preset condition.

As another example, in a CT imaging process performed to the back of the object, a prostrate posture on the scanning table 113 may be suitable. In this case, after determining the location information of the object, the processing device 140 may further determine the posture of the object based on the skeleton information. In some embodiments, the skeleton identification model may make a distinction between the patient's back and abdomen. For example, when the patient lies supine on the scanning table 113, the skeleton identification model may output a skeleton map corresponding to the supine posture that is displayed in the red color. As another example, when the patient lies on the scanning table 113 in the prostrate posture, the skeleton identification model may output a skeleton map corresponding to the prostrate posture that is displayed in the green color. When the processing device 140 determines that the posture of the object is the prostrate posture, the processing device 140 may determine that the posture of the object satisfies the preset condition.

As still another example, in a CT imaging process performed to the abdomen of the object, a supine posture on the scanning table 113 may be suitable.

As still another example, if the object is a baby, when the baby is crying, the component of the baby that is to be scanned may be in a moving state, which may lead to a lower quality of the medical image of the component. In this case, the processing device 140 may further determine the motion state of the object. In some embodiments, the processing device 140 may obtain a video of the baby through a camera and analyze the video. When the processing device 140 determines that the location of a specific component of the baby is not changed during a period of time (e.g., 0.5 s, 1 s, 3 s, 5 s, 10 s, etc.) in the video, the processing device 140 may determine that the baby is in the static state, and determine that the motion state of the baby satisfies the preset condition.

In some embodiments, if a scan is needed to be performed to the chest of the object, the processing device 140 may further determine the respiration state of the object. When the processing device 140 determines that the location, the posture, and the motion state of the object satisfy the preset condition, if the processing device 140 determines that the respiration state of the object is in a state of holding breath, the processing device 140 may determine that the respiration state of the object satisfies the preset condition, and may determine the current time as the exposure moment.

In some embodiments, the processing device 140 may determine whether the at least one of the location information of the object, the posture of the object, and the motion state of the object satisfies the preset condition using a trained machine learning model.

Taking determining whether the motion state of the object satisfies the preset condition as an example, the preset condition may include a condition that a specific component of the object is in the static state. For example, when a scan is needed to be performed to the catagmatic right hand of a patient, after determining the location information of the right hand of the patient (e.g., the processing device 140 may determine one or more mark points representing the right hand's joints using the skeleton identification model), the processing device 140 may generate a motion threshold of the right hand's joints using the trained machine learning model. The motion threshold may be used to determine whether the patient's right hand is in the static state. The processing device 140 may determine the location information of the mark points representing the right hand's joints using the skeleton identification model in a video of the object. When the skeleton identification model determines that the variation, in two or more consecutive frames in the video, of the location information of the mark points representing the right hand's joints exceeds the motion threshold, the skeleton identification model may determine that the right hand of the patient is in the moving state. When the determined motion state (e.g., the moving state) is input to another trained machine learning model, the trained machine learning model may determine that the motion state does not satisfy the preset condition (e.g., the current time may not be determined as the exposure moment). When the skeleton identification model determines that the variation, in two or more consecutive frames in the video, of the location information of the mark points representing the right hand's joints is less than the motion threshold, the skeleton identification model may determine that the right hand of the patient is in the static state. When the determined motion state (e.g., the static state) is input to another trained machine learning model, the trained machine learning model may determine that the motion state satisfies the preset condition (e.g., the current time may be determined as the exposure moment).

In some embodiments, the trained machine learning model used to determine whether at least one of the location information, the posture, and the motion state satisfies the preset condition may process the location information, the posture, and the motion state of the object and output a result as to whether at least one of the location information, the posture, and the motion state satisfies the preset condition (e.g., whether the current time is suitable to be determined as the exposure moment). The trained model may be a classification model. In some embodiments, a plurality of positive samples and negative samples may be obtained. The positive samples may include the location information, the postures, and the motion states of a plurality of sample objects that satisfy the preset condition. The negative samples may include the location information, the postures, and the motion states of a plurality of sample objects that do not satisfy the preset condition. A preliminary model may be trained using the plurality of positive samples and negative samples to obtain the trained machine learning model with a function of determining whether at least one of the location information, the posture, and the motion state of the object satisfies the preset condition.

In some embodiments, the preset condition may be updated in real time or at intervals. In some embodiments, when the processing device 140 or a user (e.g., a doctor, a technician, an engineer, etc.) of the medical radiation system 100 determines the current time as an exposure moment of a radiation process (e.g., a scan process or a radiotherapy process), the object information of the object in the radiation process (e.g., at least one of the location, the posture, and the motion state determined based on the image information of the object) may be marked as positive samples, and the object information and the marked result may be stored. When the classification model is put into use, a subsequent training process may be performed to the classification model to update the classification model at intervals, which may improve the accuracy of determining the exposure moment. In the subsequent training process, the object information of the object in the radiation process may be used as the supplementary positive samples.

In 820, in response to a determination that the at least one of the location information of the object, the posture of the object, and the motion state of the object satisfies the preset condition, the processing device 140 (e.g., the exposure moment determination module 430) may determine the exposure moment. For example, the processing device 140 may determine the current time as the exposure moment.

In some embodiments, in response to a determination that at least one of the location information of the object, the posture of the object, and the motion state of the object does not satisfy the preset condition, the processing device 140 (e.g., the exposure moment determination module 430) may generate prompting information to facilitate the satisfying of the preset condition. In some embodiments, the processing device 140 may directly output the prompting information, e.g., display a text, output a prompting voice, output a prompting sound, or the like, or any combination thereof. In some embodiments, the processing device 140 may transmit the prompting information to the terminal 130 related to the patient and/or the doctor of the medical radiation system 100. For example, when the processing device 140 determines that the location information of the patient does not satisfy the preset condition, the processing device 140 may output the prompting information that requires the patient to adjust the location of the component to be scanned to make the location of the component to be scanned satisfy the preset condition. Alternatively or additionally, the processing device 140 may adjust the location information of the scanning table 113 to make the location of the component to be scanned satisfy the preset condition. As another example, when the processing device 140 determines that the posture of the patient does not satisfy the preset condition, the processing device 140 may output the prompting information that requires the patient to adjust his/her posture to make the posture of the patient satisfy the preset condition. As still another example, when the processing device 140 determines that the component of the patient to be scanned is in the moving state, the processing device 140 may output the prompting information that requires the patient to keep his/her component to be scanned static.

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure.

FIG. 9 is a flowchart illustrating an exemplary process for generating a skeleton identification model according to some embodiments of the present disclosure. The operations of the illustrated process presented below are intended to be illustrative. In some embodiments, the process 900 may be accomplished with one or more additional operations not described, and/or without one or more of the operations discussed. Additionally, the order in which the operations of the process 900 as illustrated in FIG. 9 and described below is not intended to be limiting.

In 910, sample image information relating to a plurality of sample objects may be obtained.

In some embodiments, the sample image information may include a video image and/or a medical image, such as a CT image, a DR image, or the like. There may be one or more sample objects in one video image. In some embodiments, the sample image information may be represented by an eigenvalue, such as the grayscale value, the color value, the texture eigenvalue, the edge feature, and the matrix, histogram, principal component obtained by transforming and/or processing the sample image information. In some embodiments, a feature map of the sample image information including image feature information may be obtained by extracting image features from the sample image information using a trained deep neural network with a body feature recognition capability. It may be understood that the sample image information may be pre-processed using the body recognition models. The intermediate output or final output of the body recognition models may be used as the feature information of the sample image information, which may improve the efficiency and accuracy of the generation of the skeleton identification model.

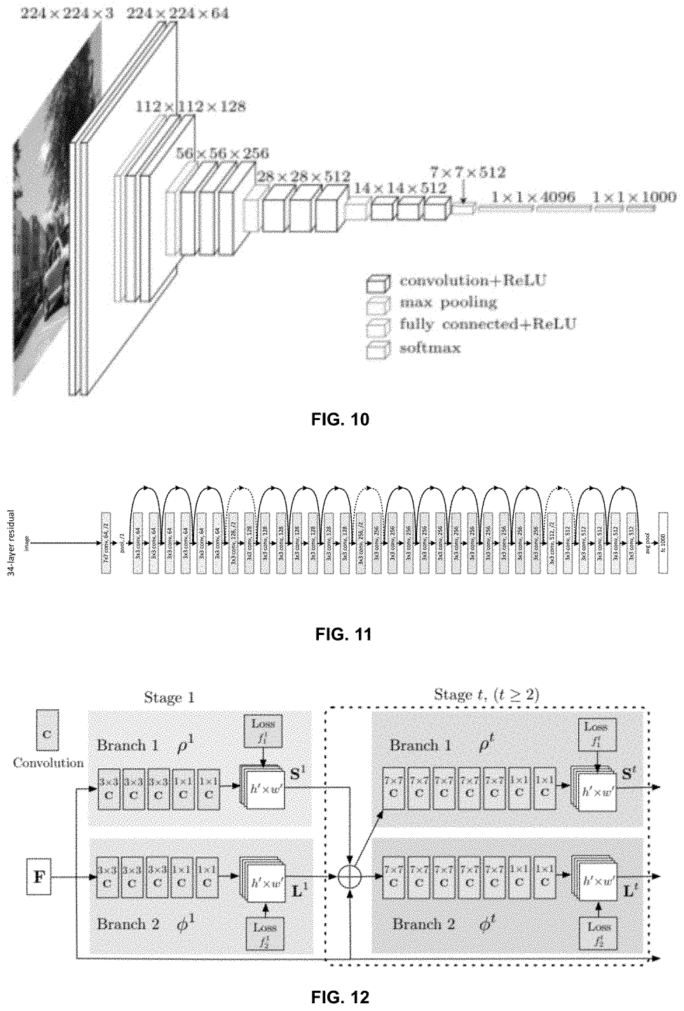

In some embodiments, the feature information of the sample image information may be extracted using the trained VGG-16 convolution Neural Network model (e.g., see, K. Simonyan., et al, In ICLR, 2015) in FIG. 10 and/or the ResNet convolution Neural Network model (e.g., see, He K, Zhang X, Ren S, et al. Deep Residual Learning for Image Recognition [J]. Computer vision and pattern recognition, 2016: 770-778) in FIG. 11.

In 920, mark points and body vectors of the plurality of sample objects in the sample image information may be obtained.

In some embodiments, the user of the medical radiation system 100 may manually mark the mark points and the body vectors in the sample image information. In some embodiments, the mark points and the body vectors may be automatically obtained using an image feature extraction algorithm. Exemplary feature extraction algorithm may include a feature extraction algorithm of histogram of oriented gradient (HOG), a feature extraction algorithm of local binary pattern (LBP), a Haar feature extraction algorithm, a feature extraction algorithm of logarithm (Log), a Harris corner feature extraction algorithm, a feature extraction algorithm of scale-invariant feature transform (SIFT), a feature extraction algorithm of speed up robust features (SURF), or the like, or any combination thereof. In some embodiments, the mark points and the body vectors in the sample image information may be obtained by processing the sample image information using one or more trained models.

In 930, the skeleton identification model may be obtained by training a preliminary model using the mark points and the body vectors in the sample image information.

In some embodiments, the preliminary model may be an iterative convolutional neural network (CNN) (e.g., see, Z. Cao, T. Simon, S.-E. Wei, and Y. Sheikh. In CVPR, 2017). The entire network architecture may be formed by linking a plurality of sub-CNN networks, for example, two or more sub-CNN networks. Through the training of a large number of samples, the value of the parameter in each sub-CNN network may be determined, and the trained skeleton identification model may be obtained.

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. For example, the iterative convolutional neural network may be replaced with other machine learning models, such as a Naive Bayesian classifier algorithm, a K-means clustering algorithm, a support vector machine learning algorithm, an Apriori machine learning algorithm, a linear regression machine learning algorithm, a Decision Tree machine learning algorithm, a random forest machine learning algorithm, a logistic regression algorithm, or the like. In some embodiments, the convolutional neural networks shown in FIG. 10 and FIG. 11 may be trained based on the process 900 to have the ability to identify the mark points and the body vectors.

In some embodiments, at least two of the trained model with a function of identifying mark points and body vectors (e.g., the skeleton identification model) the trained model with a function of determining the skeleton map, the trained model with a function of determining at least one of the location information, the posture, the motion state, and the respiration state based on the skeleton map, and the trained model with a function of determining whether at least one of the location information, the posture, and the motion state satisfies the preset condition disclosed in the present disclosure may be combined as a single model.

In some embodiments, the generation process (e.g., the process 900) of the trained models disclosed in the present disclosure may be performed by the processing device 140 or an external device communicating with the medical radiation system 100.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and/or “some embodiments” mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures or characteristics may be combined as suitable in one or more embodiments of the present disclosure.

Further, it will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “unit,” “module,” or “system.” Furthermore, aspects of the present disclosure may take the form of a computer program product embodied in one or more computer readable media having computer readable program code embodied thereon.

A non-transitory computer readable signal medium may include a propagated data signal with computer readable program code embodied therein, for example, in baseband or as part of a carrier wave. Such a propagated signal may take any of a variety of forms, including electro-magnetic, optical, or the like, or any suitable combination thereof. A computer readable signal medium may be any computer readable medium that is not a computer readable storage medium and that may communicate, propagate, or transport a program for use by or in connection with an instruction execution system, apparatus, or device. Program code embodied on a computer readable signal medium may be transmitted using any appropriate medium, including wireless, wireline, optical fiber cable, RF, or the like, or any suitable combination of the foregoing.

Computer program code for carrying out operations for aspects of the present disclosure may be written in any combination of one or more programming languages, including an object oriented programming language such as Java, Scala, Smalltalk, Eiffel, JADE, Emerald, C++, C#, VB. NET, Python or the like, conventional procedural programming languages, such as the “C” programming language, Visual Basic, Fortran 2003, Perl, COBOL 2002, PHP, ABAP, dynamic programming languages such as Python, Ruby and Groovy, or other programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider) or in a cloud computing environment or offered as a service such as a Software as a Service (SaaS),

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various inventive embodiments, This method of disclosure, however, is not to be interpreted as reflecting an intention that the claimed subject matter requires more features than are expressly recited in each claim. Rather, inventive embodiments lie in less than all features of a single foregoing disclosed embodiment.

In some embodiments, the numbers expressing quantities, properties, and so forth, used to describe and claim certain embodiments of the application are to be understood as being modified in some instances by the term “about,” “approximate,” or “substantially.” For example, “about,” “approximate,” or “substantially” may indicate ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable,

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that may be employed may be within the scope of the application. Thus, by way of example, but not of limitation, alternative configurations of the embodiments of the application may be utilized in accordance with the teachings herein. Accordingly, embodiments of the present application are not limited to that precisely as shown and described.

1. A system for exposure controlling in medical device, comprising:
at least one storage device including a set of instructions, or the set of instructions and preset data;
at least one processor in communication with the at least one storage device, wherein when executing the set of instructions, the at least one processor is directed to cause the system to perform operations including:
obtaining one or more exposure parameters relating to an exposure process associated with an abject performed by a radiation device;
obtaining object information relating to the object;
determining an exposure moment based on the object information; and
causing the radiation device to perform the exposure process to the object based on the one or more exposure parameters and the exposure moment.

2. The system of claim wherein the object information includes at least one of a position of the object, a posture of the object, and a motion state of the object.

3. The system of claim 2, wherein to obtain the object information relating to the object, the at least one processor is directed to cause the system to perform the operations including:
obtaining image information of the object, wherein the image information is provided by an image capture device;
obtaining a first trained machine learning model: and
obtaining the object information by processing the image information using the first trained machine learning model.

4. (canceled)

5. The system of claim 3, wherein to obtain the object information related to the object, the at least one processor is directed to cause the system to perform the operations including:
determining skeleton information of the object based on the image information of the object; and
determining the at least one of the position of the object, the posture of the object, and the motion state of the object based on the skeleton information.

6. The system of claim 5, wherein the skeleton information of the object is determined using the first trained machine learning model based on the image information of the object.

7. The system of claim 6, wherein the first trained machine learning model is provided by
obtaining sample image information relating to a plurality of sample objects;
obtaining mark points and body vectors of the plurality of sample objects in the sample image information, each body vector linking two of the mark points; and
obtaining the first trained machine learning model by training a preliminary model using the mark points and the vectors of the sample object.

8. The system of claim 3, wherein the first trained machine learning model is a neural network.

9. The system of claim 2, wherein to determine the exposure moment based on the object information, the at least one processor is directed to cause the system to perform the operations including:
determining whether the at least one of the position of the object, the posture of the object, and the motion state of the object satisfies a preset condition; and
in response to a determination that the at least one of the position of the object, the posture of the object, and the motion state of the object satisfies the preset condition, determining the exposure moment.

10. The system of claim 9, wherein a determination result as to whether the at least one of the position of the object, the posture of the object, and the motion state of the object satisfies the preset condition is obtained using a second trained machine learning model.

11. The system of claim 2, wherein the motion state of the object includes at least one of a motion state of the object's body and a respiration state of the object.

12. A system for exposure controlling in medical device, comprising:
one or more image capture devices configured to obtain image information of an object, wherein the image information is processed to determine object information of the object, and the object information is processed to determine an exposure moment at which a radiation device perform an exposure process to the object.

13. A method for exposure controlling in medical device implemented on a machine having one or more processors and one or more storage devices, the method comprising:
obtaining one or more exposure parameters relating to an exposure process associated with an object performed by a radiation device;
obtaining object information relating to the object;
determining an exposure moment based on the object information; and
causing the radiation device to perform the exposure process to the object based on the one or more exposure parameters and the exposure moment.

14. The method of claim 13, wherein the object information includes at least one of a position of the object, a posture of the object, and a motion state of the object.

15. The method of claim 14, wherein the obtaining the object information relating to the object includes:
obtaining image information of the object, wherein the image information is provided by an image capture device;
obtaining a first trained machine learning model; and
obtaining the object information by processing the image information using the first trained machine learning model.

16. (canceled)

17. The method of claim 15, wherein the obtaining the object information related to the object includes:
determining skeleton information of the object based on the image information of the object: and
determining the at least one of the position of the object, the posture of the object, and the motion state of the object based on the skeleton information.

18. The method of claim 17, wherein the skeleton information of the object is determined using the first trained machine learning model based on the image information of the object.

19. The method of claim 18, wherein the first trained machine learning model is provided by
obtaining sample image information relating to a plurality of sample objects;
obtaining mark points and body vectors of the plurality of sample objects in the sample image information, each body vector linking two of the mark points; and
obtaining the first trained machine learning mod& by training a preliminary model using the mark points and the vectors of the sample object.

20. (canceled)

21. The method of claim 14, wherein the determining the exposure moment based on the object information includes:
determining whether the at least one of the position of the object, the posture of the object, and the motion state of the object satisfies a preset condition; and
in response to a determination that the at least one of the position of the object, the posture of the object, and the motion state of the object satisfies the preset condition, determining the exposure moment.

22. The method of claim 21, wherein a determination result as to whether the at least one of the position of the object, the posture of the object, and the motion state of the object satisfies the preset condition is obtained using a second trained machine learning model.

23. The method of claim 14, wherein the motion state of the object includes at least one of a motion state of the object's body and a respiration state of the object.

24-35. (canceled)
